(19) United States
(12) Patent Application Publication
(10) Pub. No.: US 2010/0240552 A1
(43) Pub. Date: Sep. 23, 2010

(54) METHOD FOR EVALUATION OF DRUG SENSITIVITY BY ANALYSIS OF POMC GENE

(75) Inventors: Kazutaka Ikeda, Tokyo (JP); Shinya Kasai, Tokyo (JP); Masakazu Hayashida, Tokyo (JP); Susumu Higuchi, Kanagawa (JP)

Correspondence Address: DARBY & DARBY P.C., P.O. BOX 770, Church Street Station, New York, NY, 10008-0770, US

(73) Assignee: Tokyo Metropolitan Organization for Medical Research, Tokyo (JP)

(21) Appl. No.: 12/597,028

(22) PCT Filed: Apr. 25, 2008
(86) PCT No.: PCT/JP2008/058083

§ 371 Date: May 28, 2010

(30) Foreign Application Priority Data
Apr. 25, 2007 (JP) 2007-114968

(51) Int. Cl. C12Q 1/68; C07H 21/04; C40B 40/06

(52) U.S. Cl. 506/16; 536/23.1; 435/6

(57) ABSTRACT

The present invention provides a method for predicting the difference in drug sensitivity among individuals by using a genetic polymorphism of the POMC gene. Specifically, the present invention provides a method for evaluating drug sensitivity, comprising associating a genetic polymorphism of POMC gene with an individual drug sensitivity.

This is the U.S. National Phase Application under 35 U.S.C. §371 of International Patent Application No. PCT/JP2008/058083 filed Apr. 25, 2008, which claims the benefit of Japanese Patent Application No. 2007-114968 filed Apr. 25, 2007, both of which are incorporated by reference herein. The International Application was published in Japanese on Nov. 6, 2008 as WO 2008/133329 A1 under PCT Article 21(2).

### FIELD OF THE INVENTION

The present invention relates to a method for evaluating drug sensitivity by analyses of the POMC gene. More particularly, the present invention relates to a method for evaluating drug sensitivity by analyses of a genetic polymorphism in the 5′ flanking region of the POMC gene.

### BACKGROUND OF THE INVENTION

Pain is a pathological condition frequently seen in various medical settings. Pain resulting from a disease or pain induced by treatment of a disease is a very serious problem for patients. Although pain sensation plays an important role as a biological warning system, undue pain could cause a significant reduction of quality of life. Recently, the importance of pain control has been recognized, and palliative care, such as pain therapy, has made significant progress in which the opportunity to use and the amount of use of various analgesics has increased. However, because the analgesic effects and side effects of such analgesics vary greatly among individuals, predicting the analgesic effect is very important to perform an optimal administration schedule for each individual.

The proopiomelanocortin (hereinafter referred to as POMC) gene encodes several peptides, including β-endorphin, one of the endogenous opioid peptides. Through behavioral pharmacological studies using POMC-deficient mice, β-endorphin and the POMC gene have been found to produce analgesic effects on nociceptive behaviors induced by various stimuli and to play an important role in morphine efficacy (Cavun, S. et al., Glycyl-glutamine, an endogenous β-endorphin-derived peptide, inhibits morphine-induced conditioned place preference, tolerance, dependence, and withdrawal. J. Pharmacol. Exp. Ther. 2005, 315:949-58; Lee, T. H. et al., In vivo electroporation of proopiomelanocortin induces analgesia in a formalin-injection pain model in rats. Pain 2003, 104:159-67; Rubinstein, M. et al., Absence of opioid stress-induced analgesia in mice lacking beta-endorphin by site-directed mutagenesis. Proc. Natl. Acad. Sci. U.S.A. 1996, 93:3995-4000). Furthermore, POMC genetic polymorphisms and haplotypes have been reported to be associated with blood leptin concentration, body fat percentage, and bone density (Bienertova-Vasku, J. A. et al., Proopiomelanocortin gene: a new susceptibility gene for cutaneous T-cell lymphoma. Proc. Eur. Soc. Dermatol. Res. 2006, S33; Baker, M. et al., Association between common polymorphisms of the proopiomelanocortin gene and body fat distribution: a family study. Diabetes 2005, 54:2492-6; Chen, Y. et al., Proopiomelanocortin gene variants are associated with serum leptin and body fat in a normal female population. Eur. J. Hum. Genet. 2005, 13:772-80; Sudo, Y. et al., Association of single nucleotide polymorphisms in the promoter region of the pro-opiomelanocortin gene (POMC) with low bone mineral density in adult women. J. Hum. Genet. 2005, 50:235-40; Suviolahti, E. et al., Pro-opiomelanocortin gene is associated with scrum leptin levels in lean but not in obese individuals. Int. J. Obes. Relat. Metab. Disord. 2003, 27:1204-11; Hixson, J. E. et al., Normal variation in leptin levels is associated with polymorphisms in the proopiomelanocortin gene, POMC. J. Clin. Endocrinol. Metab. 1999, 84:3187-91). Additionally, morphine sensitivity significantly varies among individuals, and the genetic mechanisms involved in individual differences in morphine sensitivity are becoming more clear (Ikeda, K. et al., How individual sensitivity to opiates can be predicted by gene analyses. Trends Pharmacol. Sci. 2005, 26:311-7).

### SUMMARY OF THE INVENTION

The objective of the present invention is to provide a method for predicting drug sensitivity in each individual, specifically drug sensitivity in each individual with respect to the required number of analgesic doses, the total amount of analgesics, and vulnerability to alcohol dependence, using POMC genetic polymorphisms.

We focused on the POMC gene and have conducted extensive examinations based on conventional findings and clinical data. As a result, we identified a useful genetic polymorphism by analyzing the association between each POMC genetic polymorphism and sensitivity to drugs, such as analgesics and alcohol. Specifically, we found that the required number of analgesic doses and the total amount of analgesics differ between genotypes in a certain POMC genetic polymorphism, specifically a genetic polymorphism in the 5′ flanking region of the POMC gene. We also found that the frequency of certain POMC genetic polymorphisms differs between alcoholic patients and healthy subjects, thereby accomplishing the present invention.

Thus, the present invention relates to the following:

(1) A method for evaluating drug sensitivity, comprising associating a genetic polymorphism in the 5′ flanking region of POMC gene with an individual drug sensitivity.

In the method according to (1) above, a drug may be, for example, a μ-opioid receptor-modifying agent. Examples of μ-opioid receptor-modifying agents include at least one selected from the group consisting of methamphetamine, methylenedioxymethamphetamine, amphetamine, dextroamphetamine, dopamine, cocaine, heroin, morphine, hydromorphone, oxymorphone, DAMGO, codeine, dihydrocodeine, methadone, oxycodone, hydrocodone, fentanyl, carfentanil, tramadol, meperidine, dextropropoxyphene, dextromoramide, levorphanol, etorphine, dihydroetorphine, buprenorphine, nalorphine, nalbuphine, pentazocine, levallorphan, butorphanol, naltrexone, alvimopan, naloxone, methanol, ethanol, propanol, butanol, pentanol, and diethyl ether.

In the method according to (1) above, examples of the gene polymorphism include at least one selected from the group consisting of a single nucleotide polymorphism, an insertion polymorphism, a deletion polymorphism, and a nucleotide repeat polymorphism. Herein, an example of such a genetic polymorphism includes a polymorphism designated by an rs number, rs3754860 (see Table 1 below).

(2) A method for determining the type, the number of doses, and/or the dosage of a drug to be administered to an individual, comprising using the results from the evaluation by the method according to (1) above as an index.

(3) A method for predicting a side effect of a drug to be administered to an individual, comprising using the results from the evaluation by the method according to (1) above as an index.

(4) An oligonucleotide comprising a nucleotide sequence of at least 10 nucleotides, including the 51st nucleotide of the nucleotide sequence represented by SEQ ID NO:1 or 2, or a complementary nucleotide sequence thereto. Herein, the oligonucleotide may, for example, span a length of 10-45 nucleotides.

(5) An oligonucleotide comprising a nucleotide sequence represented by SEQ ID NO:1 or 2 or a complementary nucleotide sequence thereto.

(6) A microarray comprising the oligonucleotide according to (4) or (5) above immobilized on a support.

(7) A kit for evaluating drug sensitivity, comprising the oligonucleotide according to (4) or (5) above and/or the microarray according to (6) above.

The present invention is able to provide a POMC genetic polymorphism (specifically, a genetic polymorphism in the 5′ flanking region of the POMC gene) that allows evaluation of the difference in drug sensitivity among individuals and a method for evaluating drug sensitivity by using this genetic polymorphism. This evaluation method allows one to easily determine the optimal dosage and schedule to be prescribed for a narcotic analgesic, such as morphine, which would be extremely useful for personalized pain therapy and drug addiction treatment.

### BRIEF DESCRIPTION OF THE DRAWINGS

FIG. 1 is a schematic view showing the POMC genetic polymorphisms used for the analysis of the invention. In the figure, the black boxes represent translated regions of the gene, and the white boxes represent untranslated regions of the gene. The arrows indicate the positions of the respective polymorphisms used for the analysis.

FIG. 2 is a graph showing the effects of the genotypes of a POMC genetic polymorphism (G-1695A) on the required number of analgesic doses during the 24 hour period following surgery (average of males and females±standard error). In the figure, “Ave.” refers to an average value of males and females, and “S.E.” refers to the standard error (which likewise applies to the other figures contained herein).

FIG. 3 is a graph showing the effects of the genetic alleles of a POMC genetic polymorphism (G-1695A) on the required number of analgesic doses during the 24 hour period following surgery (average of males and females±standard error).

FIG. 4 is a graph showing the effects of the genotypes of a POMC genetic polymorphism (IVS1+C266G) on the required number of analgesic doses during the 24 hour period following surgery (average of males and females±standard error).

FIG. 5 is a graph showing the effects of the genotypes of a POMC genetic polymorphism (TGA+C63T) on the required number of analgesic doses during the 24 hour period following surgery (average of males and females±standard error).

FIG. 6 is a graph showing the effects of the genotypes of a POMC genetic polymorphism (G-1695A) on the total amount of analgesics converted to pentazocine-equivalent dosages (average of males and females±standard error).

FIG. 7 is a graph showing the effects of the genetic alleles of a POMC genetic polymorphism (G-1695A) on, the total amount of analgesics converted to pentazocine-equivalent dosages (average of males and females±standard error).

FIG. 8 is a graph showing the effects of the genotypes of a POMC genetic polymorphism (IVS1+C266G) on the total amount of analgesics converted to pentazocine-equivalent dosages (average of males and females±standard error).

FIG. 9 is a graph showing the effects of the genotypes of a POMC genetic polymorphism (TGA+C63T) on the total amount of analgesics converted to pentazocine-equivalent dosages (average of males and females±standard error).

### DETAILED DESCRIPTION OF THE INVENTION

Hereinafter, the present invention will be described in detail. The scope of the invention should not be limited to the descriptions below, and the invention may appropriately be modified and carried out in ways other than the following examples without departing from the spirit of the invention.

The present specification incorporates the entire content of the specification of Japanese Patent Application Publication No. 2007-114968, based on which the present application claims priority. All of the literature cited herein, for example, prior art documents, laid-open patent publications, patent publications, and other patent documents, are incorporated herein by reference.

1. Outline of the Invention

(1) POMC (Proopiomelanocortin)

Narcotic analgesics such as morphine act on opioid receptors to induce analgesia. Three types of opioid receptors, mu (μ), delta (δ), and kappa (κ), are involved in analgesia. A number of endogenous opioid peptides that act on these opioid receptors are present in an organism. β-endorphin, one of these endogenous opioid peptides, provides analgesia. Although behavioral pharmacological studies using β-endorphin, as well as agonists and antagonists of respective opioid receptors, have been reported, the mechanism of action of β-endorphin on opioid receptors has not yet been confirmed.

Here, the POMC gene will be described. The POMC gene encodes an opioid peptide, β-endorphin, as well as nonopioid peptides, adrenocorticotropin (adrenocorticotropic hormone, ACTH), corticotropin-like intermediate-lobe peptide (CLIP), β- and γ-lipotropins (LPHs), and α-, β-, and γ-melanotropins (melanocyte stimulating hormones, MSHs). First, a precursor peptide is synthesized from the POMC gene, which is cleaved by protease at respective regions of the brain to yield respective peptides. In the anterior pituitary, ACTH, γ-MSH, β-LPH, as well as breakdown products of β-LPH (i.e., γ-LPH and β-endorphin) are yielded. β-Endorphin is further broken down into β-endorphin₁₋₂₇ which diminishes the analgesic effect of β-endorphin. In the anterior pituitary, ACTH is further broken down into α-MSH and CLIP, and γ-LPH yields β-MSH. In POMC-deficient mice, phenotypes such as degradation of coat/hair pigmentation, obesity, reduction of various hormones, and reduction of epinephrine production are observed, whereas a phenotype such as failure of stress-induced analgesia is observed in β-endorphin-deficient mice. In humans, POMC-deficient patients display ACTH deficiency, which eventually causes adrenal cortical hypofunction that progresses to death without ACTH administration.

(2) POMC Genetic Polymorphisms and Drug Sensitivity

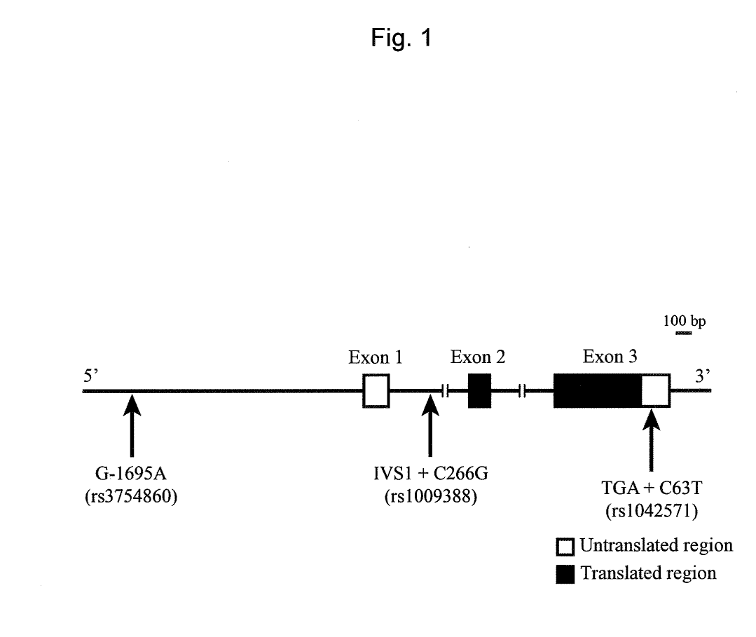

We analyzed genetic polymorphisms (single-nucleotide polymorphisms [SNPs], etc.) of the POMC gene, which have been identified to be significantly different in other diseases. The analyzed POMC genetic polymorphisms were the three polymorphisms in the 5′ flanking region, intron 1, and 3′ untranslated region (see FIG. 1). Among these polymorphisms, one with a higher minor allele frequency was G-1695A (rs number: rs3754860) in the 5′ flanking region of the POMC gene (see Table 1), which was subsequently analyzed.

We determined the genotype of one of the POMC genetic polymorphisms (G-1695A) for patients who underwent surgery. Analyses of the associations between the genotype of the POMC genetic polymorphism and the required number of analgesic doses and the total amount of analgesics following surgery showed that the required number of analgesic doses and the total amount of analgesics were significantly increased for a patient group with the A/A genotype of this polymorphism compared with a patient group with the G/A genotype. Additionally, the required number of analgesic doses and the total amount of analgesics were significantly increased for a patient group without the G allele of this polymorphism compared with a patient group with the G allele. In IVS1+C266G and TGA+C63T polymorphisms, however, no significant differences were found with regard to the required number of analgesic doses and the total amount of analgesics after surgery between the patients of the respective genotypes.

Furthermore, the genotype of the POMC genetic polymorphism (G-1695A) was determined for alcoholic patients and healthy subjects. As a result, the G/G and G/A genotype frequencies were significantly higher for the patient group compared with the 1*1 healthy subject group. A significant difference in the genotype frequency of the G-1695A polymorphism was found between alcoholic patients and healthy subjects with the 1*1 genotype of the ALDH2 gene, in which the alcoholic patients had significantly high G/G and G/A genotype frequencies. The G/G and G/A genotype frequencies of the G-1695A polymorphism were significantly higher for alcoholic patients with the 1*2 genotype of the ADH2 gene than healthy subjects with the 1*2 genotype of the ADH2 gene.

Moreover, methamphetamine-dependent patients with prolonged stimulant psychosis displayed a tendency toward higher G/G and G/A genotype frequencies for the G-1695A polymorphism compared with patients who underwent early withdrawal.

The G-1695A genetic polymorphism (rs3754860) in the 5′ flanking region of the POMC gene appears to affect the promoter activity of the POMC gene, and the A/A genotype is expected to reduce the expression of the POMC gene.

Hence, an analysis of the genetic polymorphism of the POMC gene identified by the invention allows easy evaluation of differences among individuals with respect to the required number of analgesic doses, total amount of analgesics, and drug sensitivity, such as vulnerability to drug addiction. This drug sensitivity would be key information for determining the type, the number of doses and the dosages of drugs. In particular, because morphine, alcohol, and so on may cause serious social issues depending on usage and the amount used thereof, predicting the appropriate dosage for each individual before administration is very important. Moreover, because abuse of stimulant-type drugs, such as methamphetamine, is rapidly increasing and recently emerging as a serious social issue, predicting the risk of stimulant drug addiction is also important. Thus, the present invention would be quite useful for personalized pain therapy and drug addition treatment.

2. POMC Genetic Polymorphisms

Although a human POMC genetic polymorphism of the invention primarily comprises, but is not limited to, SNPs, it may also comprise insertion/deletion polymorphisms and polymorphisms resulting from various nucleotide repeats.

An SNP refers to a polymorphism that results from substitution of a particular single nucleotide of a gene with another nucleotide. An insertion/deletion polymorphism refers to a polymorphism resulting from insertion/deletion of one or more nucleotides. Additionally, a polymorphism (a nucleotide repeat polymorphism) resulting from various nucleotide repeats may be a microsatellite polymorphism (number of nucleotide repeats: approximately 2-7 nucleotides) or a variable number of tandem repeat (VNTR) polymorphism (number of nucleotide repeats: several to several tens of nucleotides) according to the difference in the number of nucleotide repeats, in which the number of repeats is different for each individual.

Information of the human POMC genetic polymorphisms identified by the invention is shown in Table 1 below. The information of the polymorphisms shown in Table 1 is associated with SNPs.

The present invention provides an oligonucleotide comprising a genetic polymorphism of the POMC gene shown in Table 1. A method for obtaining the information of the genetic polymorphisms of the present invention shown in Table 1 is as follows.

Genomic DNA is purified from a human blood sample by the phenol chloroform method or the like. In this regard, a commercially available genomic DNA extraction kit or device such as the GFX Genomic Blood DNA Purification Kit (GE Healthcare Japan) may be used. Next, the obtained genomic DNA is used as a template to amplify the DNA fragments in part by the polymerase chain reaction (PCR) method to obtain DNA fragments for enzymatic cleavage or sequencing. The present invention involves a polymorphism of a gene, and the DNA polymerase used for the PCR method preferably has as high fidelity as possible. According to the PCR-restriction fragment length polymorphism (PCR-RFLP) technique, primers designed based on the sequence information available from the GenBank database (hereinafter, GenBank) on the National Center for Biotechnology Information (NCBI) website (http://www.ncbi.nlm.nih.gov) are used to perform PCR amplification of about 700 nucleotides, including the polymorphisms of the POMC gene and the flanking sequences. These PCR fragments are cleaved with a particular restriction enzyme and subjected to electrophoresis with a visualizing dye in an agarose gel at an appropriate concentration. Subsequently, DNA is stained with ethidium bromide to identify the genotype of the genetic polymorphisms according to the lengths of the DNA fragments. In a sequencing procedure, primers whose nucleotide sequences are designed based on the sequence information available from GenBank are used for each polymorphism of the POMC gene to perform PCR amplification of about 700 nucleotides, including the polymorphisms and the flanking sequences. The nucleotide sequences of these PCR fragments are sequenced for about 300-700 nucleotides at a time by a sequencing procedure to obtain the genetic polymorphism of interest. As an example of the resulting polymorphism information, exemplary SNPs and their polymorphism frequencies in the POMC gene observed in the genomes of Japanese healthy subjects are shown in Table 1 below.

TABLE 1

| Gene | | SNP | | Submitted | Major | Minor | Minor allele | Sample |
|---|---|---|---|---|---|---|---|---|
| Name | Region | name | rs number | allele | allele | allele | frequencies | size |
| POMC | 5′ flanking | G-1695A | rs3754860 | G | G | A | 0.324 | 139 |
| | region | | | | | | | |
| | Intron 1 | IVS1 + | rs1009388 | C | C | G | 0.032 | 139 |
| | | C266G | | | | | | |
| | 3′ untranslated | TGA + | rs1042571 | C | C | T | 0.011 | 139 |
| | region | C63T | | | | | | |

In Table 1, the term “gene name” (POMC) refers to the human POMC gene.

The term “position” refers to positions on the genome of the POMC gene, such as the 5′ flanking region, intron 1, and 3′ untranslated region.

The term “SNP name” is the name of SNPs named by the present inventors after the positions on the POMC gene. Basically, the name includes notation of a 2-5 digit number and signs, with letters A, G, C, or T at both ends to identify the nucleotides of the SNP. For example, “G-1695A” along the row “5′ flanking region” indicates that the nucleotide located at 1695 nucleotides upstream (5′ direction) of the nucleotide immediately preceding exon 1 is a polymorphism of G to A substitution. Here, “G-1695A” is an SNP name given by the present inventors to the genetic polymorphism with the rs number (described later in detail) rs3754860 with reference to a contig sequence of human chromosome 2 (Accession number: NT_{—}022184) registered with the GenBank database available on the NCBI (the National Center for Biotechnology Information) website (http://www.ncbi.nlm.nih.gov). Additionally, the polymorphism in the complementary strand corresponding to this polymorphism (G-1695A) is called by the SNP name “C-1802T” (Chen, Y. et al., Eur. J. Hum. Genet. 2005, 13:772-80; op. cit.), and thus G-1695A and C-1802T refer to substantially the same polymorphism. “C-1802T” indicates that the nucleotide located at 1802 nucleotides upstream (5′ direction) of the transcription initiation site of the POMC gene is a polymorphism of C to T substitution. Moreover, “IVS1+C266G” along the row “Intron 1” is a polymorphism in intron 1 and indicates that the nucleotide located at 266 nucleotides downstream (3′ direction) of the nucleotide at the starting point of intron 1 (IVS1) is a polymorphism of C to G substitution. Also, “TGA+C63T” along the row “3′ untranslated region” indicates that the nucleotide located at 63 nucleotides downstream (3′ direction) of the nucleotide following the translation stop site (TGA) is a polymorphism of C to T substitution.

The term “rs number” is a reference cluster ID number registered in the dbSNP database of genetic polymorphisms provided on the NCBI website (op. cit.). The number consists of “rs” followed by a several-digit number, and each polymorphism identified by this number is defined by species, gene name, site, and the like. In other words, each genetic polymorphism essentially has a single assigned rs number.

The term “submitted allele” refers to an allele of the sequence registered with GenBank in which a “major allele” and a “minor allele” represent major and minor alleles, respectively, among the genomes of Japanese patients who have underwent surgery. The term “minor allele frequencies” refers to a proportion of a minor allele.

The term “sample size” refers to the number of Japanese patients as test subjects who underwent surgery.

The polymorphisms shown in Table 1 are included in the POMC genetic polymorphisms of the invention. Furthermore, oligonucleotides comprising the genetic polymorphisms of the invention are shown in Table 2. For example, an oligonucleotide of the invention is selected from the nucleotide sequences represented by SEQ ID NOS:1-6 (particularly and preferable SEQ ID NO:1 or 2), including the aforementioned polymorphisms or complementary nucleotide sequences thereto (Table 2).

TABLE 2

| | | | Direction | | |
|---|---|---|---|---|---|
| Gene | | SNP | of gene | Sequence flanking | SEQ |
| Name | Region | name | sequence | genetic polymorphism | ID NO. |
| POMC | 5′ flanking | G-1695A | Gene strand | 5′-ACCAAAACCCAAAATTAGCTGGGCATGGT | 1 |
| | region | | | GGCATGCATCTGTGGTTCCAG G/A | |
| | | | Complementary | 5′-GCCTCCACCTCCTGGGCTCAAGTGATCCT | 2 |
| | | | strand | CATGCTTCAGCCTCCTGAGTA C/T | |
| | | | | CTGGAACCACAGATGCATGCCACCATGCCCAG | |
| | | | | CTAATTTTGGGTTTTGGT-3′ | |
| | Intron 1 | IVS1 + C266G | Gene strand | 5′-CCGCGAAGCTGCAGGCGCTGTCTCCAGGG | 3 |
| | | | | AGCCGGCGGCCTCCTCTCCCC C/G | |
| | | | Complementary | 5′-CCAAGACCTCCTAGCAAGCTCTCGGAGCC | 4 |
| | | | strand | TCCGGACCGCCGCGAGCCCCT G/C | |
| | | | | GGGGAGAGGAGGCCGCCGGCTCCCTGGAGACA | |
| | | | | GCGCCTGCAGCTTCGCGG-3′ | |
| | 3′ untranslated | TGA + C63T | Gene strand | 5′-GCCCCAGGGCTACCCTCCCCCAGGAGGTC | 5 |
| | region | | | GACCCCAAAGCCCCTTGCTCT C/T | |
| | | | Complementary | 5′-CTGATTATCTGCCACGACCCCCCAGGCTG | 6 |
| | | | strand | GGAGGCGGCAGCAGGGCAGGG A/G | |
| | | | | AGAGCAAGGGGCTTTGGGGTCGACCTCCTGGG | |
| | | | | GGAGGGTAGCCCTGGGGC-3′ | |

Table 2 shows nucleotide sequences (SEQ ID NOS:1-6: Gene strand or complementary strand) consisting of 101 nucleotides, with a polymorphism site at the 51st nucleotide. For example, the annotations “G/A,” “C/T,” and “C/G” represent polymorphisms of “G” and “A,” “C” and “T,” and “C” and “G,” respectively. Among these, the present invention provides an oligonucleotide consisting of a nucleotide sequence having at least 10 nucleotides, preferably 10-100 nucleotides, more preferably 10-45 nucleotides, still more preferably 14-25 nucleotides, including the polymorphism site (the 51st nucleotide) of the nucleotide sequence shown in Table 2 (SEQ ID NOS:1-6, particularly and preferably SEQ ID NO:1 or 2) or a complementary nucleotide sequence thereto.

3. Association Between POMC Genetic Polymorphisms and Drug Sensitivity

Polymorphisms in the POMC gene appear to alter the function and expression levels of β-endorphin, adrenocorticotropin (adrenocorticotropic hormone, ACTH), corticotropin-like intermediate-lobe peptide (CLIP), β- and γ-lipotropins (LPHs), α-, β-, and γ-melanotropins (melanocyte-stimulating hormones, MSHs) and the like encoded by the POMC gene. Therefore, POMC genetic polymorphisms may be associated with various phenotypes involved with β-endorphin, ACTH, CLIP, lipotropin, and MSH. Here, examples of phenotypes may include various phenotypes relative to onsets of diseases and phenotypes relative to drug sensitivity. Examples of phenotypes relative to onsets of diseases include pain sensitivity and vulnerability to drug addiction, and examples of phenotypes relative to drug sensitivity include potency of a drug, side effects of a drug, and effective duration of a drug and the like.

The association between POMC genetic polymorphisms and phenotypes may be examined as follows. As such, the POMC genetic polymorphism, a polymorphism that has significant association with other diseases (i.e., a functional polymorphism), or a tag polymorphism in the linkage block deduced from the results from linkage disequilibrium and haplotype analyses of healthy subjects is selected (for example, an SNP). Next, the frequency of this polymorphism (e.g., SNP) in test subjects (patients) is analyzed to compare with the polymorphism frequency in healthy subjects. For comparison, statistical means such as χ² test may be effectively employed.

Where the phenotype or condition is alcohol dependence, it may be classified, for example, according to the potential of accompanying nonsocial behavior, age at dependence onset, polydrug abuse, the potential for an accompanying psychosis-like state or a state of depression, the genotype of the alcohol dehydrogenase 2 (ALDH2) gene, or the genotype of the aldehyde dehydrogenase 2 (ADH2) gene. For each classification, polymorphism frequencies or genotype frequencies are compared between healthy subjects and target subjects. A polymorphism with a significant difference in the polymorphism frequency or genotype frequency from the control group may be used to evaluate susceptibility to the disease and differences in drug sensitivity. However, because the frequency of a genetic polymorphism is suggested to be influenced by ethnic background, birthplace, and the like, the aforementioned evaluation is preferably performed with a population that shows similar frequencies of the genetic polymorphisms to the population used for identifying the predicting polymorphisms (e.g., SNPs). In this regard, the application of different data from a newly analyzed population allows analysis of the different population.

4. Application of the Analysis Results

The results from the above analysis may be used as an index in a method for predicting sensitivity to various drugs associated with the POMC gene, a method for selecting treatment or prevention of a disease associated with the POMC gene, a method for determining an appropriate dosage of a therapeutic drug, and a method for predicting side effects and the like. An oligonucleotide or a microarray of the invention may be used to determine a genotype of an individual to allow predicting and evaluating the individual's drug sensitivity.

Examples of an opioid receptor function-modifying agent as a target for evaluating sensitivity include stimulant-type drugs, such as methamphetamine, dopamine receptor agonists, dopamine receptor antagonists, μ-, δ-, and κ-opioid receptor agonists, μ-, δ-, and κ-opioid receptor antagonists, and alcohol and the like, in which a μ-opioid receptor function-modifying agent is particularly preferable. Here, specific examples of a μ-opioid receptor-modifying agent include the following: methamphetamine, methylenedioxymethamphetamine, amphetamine, dextroamphetamine, dopamine, cocaine, heroin, morphine, hydromorphone, oxymorphone, DAMGO, codeine, dihydrocodeine, methadone, oxycodone, hydrocodone, fentanyl, carfentanil, tramadol, meperidine, dextropropoxyphene, dextromoramide, levorphanol, etorphine, dihydroetorphine, buprenorphine, nalorphine, nalbuphine, pentazocine, levallorphan, butorphanol, naltrexone, alvimopan, naloxone, methanol, ethanol, propanol, butanol, pentanol, and diethyl ether.

Moreover, the genetic polymorphisms or the method of the invention may be used to evaluate drug sensitivity or the like that differs by ethnic group. Although subjects are not particularly limited and may include Japanese, American, or European or the like, subjects in the present invention are preferably Japanese or those having similar frequencies of the genetic polymorphisms to Japanese.

Genomic samples may be extracted from blood, saliva, or skin or the like of the test subjects, which are not limited thereto as long as they allow extraction of genomic DNA. Extraction and purification methods for genomic DNA are well known. For example, genomic DNA is purified from human specimens such as blood, saliva, or skin or the like by the phenol chloroform method or the like. In this regard, a commercially available genomic DNA extraction kit or device, such as the GFX Genomic Blood DNA Purification Kit, may be used. When an SNP to be analyzed is in the exon, mRNA or total RNA may be extracted instead of genomic DNA. Hereinafter, exemplary methods for detecting a genetic polymorphism of the test sample will be described.

(1) Detection by PCR Method

To amplify the test sample by PCR, a DNA polymerase with high fidelity, such as KOD Dash DNA polymerase (TOYOBO), is preferably used. The primers used are designed and synthesized such that the genetic polymorphism is included at any position of the primers that allows amplification of the target SNP in the test sample.

At the end of the amplification reaction, the amplified product is detected to determine the genotype of the genetic polymorphism.

(2) Detection by PCR-RFLP Method

A DNA polymerase with high fidelity, such as KOD Dash DNA polymerase (TOYOBO), is used to amplify the test sample by PCR. The primers used are designed and synthesized such that the target SNP in the test sample is included at any position of the amplified PCR fragments. These PCR fragments are cleaved with particular restriction enzymes and subjected to electrophoresis with a visualizing dye in an agarose gel at an appropriate concentration. Finally, DNA is stained with ethidium bromide to determine the genotype of the genetic polymorphism according to the lengths of the DNA fragments.

(3) Detection by Sequencing Procedure

According to the present invention, a polymorphism of the invention may also be detected by a sequencing procedure based on a dideoxy procedure. A commercially available ABI series (Applied Biosystems) is employed as a sequencer used for nucleotide sequences.

(4) Detection by DNA Microarray

A DNA microarray has nucleotide probes immobilized on a support, examples being a DNA chip, a gene chip, a microchip, and a bead array and the like.

First, the polynucleotide of the test sample is isolated, amplified by PCR, and labeled with fluorescent reporter molecules. Subsequently, the labeled DNA, mRNA, or total RNA are incubated with the array. Next, the hybridization pattern of this array is detected with a scanner. As hybridization data, fluorescent intensities from the fluorescent reporter molecules bound to the probe array (i.e., incorporated into the target sequence) are used. A region immobilized with a probe completely matching the target sequence gives a stronger signal than that from a region with a probe that does not match the target sequence. Because the sequence and position of each probe on the array are known, the sequence of the target polynucleotide that reacted with the probe array via complementarity can be determined.

(5) Detection by Taqman PCR Method

The TaqMan PCR method employs an allele-specific oligonucleotide labeled with a fluorescent molecule and PCR reaction using Taq DNA polymerase. The allele-specific oligonucleotide used with the TaqMan PCR method (referred to as a TaqMan probe) may be designed based on the aforementioned genetic polymorphism information.

(6) Detection of Genetic Polymorphism by Invader Assay

The invader assay is a method for detecting a genetic polymorphism by hybridizing an allele-specific oligonucleotide and a template. Use of a commercially available kit allows easy detection of the genetic polymorphism by invader assay.

5. Kit

According to the present invention, an oligonucleotide containing a POMC genetic polymorphism (e.g., SNP) site may be contained in a kit for detecting a genetic polymorphism.

The genetic polymorphism detection kit of the invention comprises one or more components required for carrying out the present invention. For example, the kit of the invention comprises a component for storing or supplying an enzyme and/or a reaction component required for carrying out genetic polymorphism detection. Examples of such components include, but are not limited to, an oligonucleotide of the invention, an enzyme buffer, dNTP, a control reagent (e.g., a tissue sample, target oligonucleotides as positive and negative controls, etc.), a labeling and/or detecting reagent, a solid support, and instructions. Alternatively, the kit of the invention may be a partial kit that contains only a part of the required components, in which case the user may prepare other components.

The kit of the invention may be provided as a microarray having the oligonucleotides immobilized on a support. The microarray has the oligonucleotide of the invention immobilized on a support, including a DNA chip, a gene chip, a microchip, and a bead array and the like.

The kit of the invention comprises an oligonucleotide comprising a POMC genetic polymorphism found by the invention. Thus, the POMC gene is isolated by a PCR method or the like by collecting blood prior to the use of a drug by a patient (e.g., before surgery or upon incidence of cancer pain) and then this gene or a fragment thereof is allowed to react with the oligonucleotide of the kit, thereby determining the genotype. The administration schedule, such as the type and dosage of drug, will be made according to the determined genotype of the genetic polymorphism. As a result, a drug effect suitable for each individual can be obtained, which is beneficial for customized medicine. For example, where morphine is used, an analgesic effect that suits each individual may be obtained while keeping side effects to a minimum.

Hereinafter, the present invention will be described in a more specific manner, although the present invention is not limited thereto.

### Example 1

### Association Between POMC Genetic Polymorphisms and Required Number of Analgesic Doses

Genomic DNA was extracted from blood or mucosa of the oral cavity of 139 patients who underwent surgery to determine the genotype of genetic polymorphisms (G-1695A polymorphism) of the POMC gene by the PCR-RFLP method with the primer sets 1-3 shown in Table 3 below (SEQ ID NOS:7-12). The primers shown in Table 4 below (SEQ ID NOS:13-16) were used to determine the genotypes of other genetic polymorphisms (IVS1+C266G and TGA+C63T polymorphisms) of the POMC gene by a sequencing procedure. Associations between the results of determination of the genotypes of these genetic polymorphisms and the required number of analgesic doses after surgery were analyzed.

TABLE 3

Primer sets for determining genotype of POMC G-1695A polymorphism
by PCR-RFLP method, lengths of amplified products and
lengths of fragments cleaved by restriction enzyme

| | | | | | | Length of |
|---|---|---|---|---|---|---|
| | | | | | | fragment |
| | | | | Length of | | cleaved by |
| Primer | | Primer sequence | SEQ | amplified | Restriction | restriction |
| sets | Direction | (length) | ID NO | product | Enzyme | enzyme |
| 1 | Forward | 5′-CCAGTGATTCTAAATGCAGTT-3′ | 7 | 517 bp | Rsa 1 | 240 bp/ |
| | primer | (21 mer) | | | | 277 bp (G) |
| | Reverse | 5′-GTAACTTCAGGAGGCTGCTGG-3′ | 8 | | | Total 517 bp |
| | primer | (21 mer) | | | | (A) |
| 2 | Forward | 5′-CCAGTGATTCTAAATGCAGTT-3′ | 9 | 388 bp | Rsa 1 | 111 bp/ |
| | primer | (21 mer) | | | | 277 bp (G) |
| | Reverse | 5′-GAATTAGGGTCTCACTCTGTTGC-3′ | 10 | | | Total 388 bp |
| | primer | (23 mer) | | | | (A) |
| 3 | Forward | 5′-CCAGTGATTCTAAATGCAGTT-3′ | 11 | 558 bp | Rsa 1 | 277 bp/ |
| | primer | (21 mer) | | | | 281 bp (G) |
| | Reverse | 5′-GCAGATGGTGGTTTTGCCTTATTT-3′ | 12 | | | Total 558 bp |
| | primer | (24 mer) | | | | (A) |

TABLE 4

Primer sets for determining genotypes of POMC
IVS1 + C266G and TGA + C63T polymorphisms by
sequencing procedure and lengths of amplified products

| | | | | Length of |
|---|---|---|---|---|
| Genetic | | Primer sequence | SEQ | amplified |
| polymorphism | Direction | (length) | ID NO | product |
| IVS1 + C266G | Forward | 5′-AGCCTCCCGAGACAGGTAAG-3′ | 13 | 578 bp |
| | primer | (20 mer) | | |
| | Reverse | 5′-CCTGTTGTCGGGAGTTTGAG-3′ | 14 | |
| | primer | (20 mer) | | |
| TGA + C63T | Forward | 5′-CCCAAGGACAAGCGCTAC-3′ | 15 | 346 bp |
| | primer | (18 mer) | | |
| | Reverse | 5′-CCATGCTGCTGTTATTTGAC-3′ | 16 | |
| | primer | (20 mer) | | |

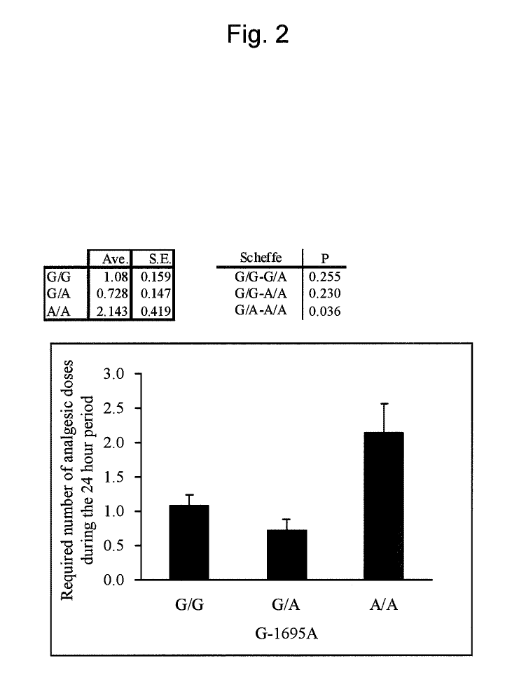

As a result, as shown in Table 5 below and FIG. 2, the patient group with the A/A genotype of the G-1695A polymorphism required a significantly greater number of analgesic doses during the 24 hour period following surgery compared with the patient group with G/A (P=0.036). In particular, females (F) with the A/A genotype tended to require a greater number of analgesic doses compared with males (M).

TABLE 5
Effects of POMC genetic polymorphisms on the number of
analgesic doses during the 24 hour period following surgery
for patients receiving analgesics upon surgery (descriptive
statistics for each genotype and sex)

| | | | | | Number |
|---|---|---|---|---|---|
| POMC genetic | | | | Standard | of test |
| polymorphism | Genotype | Sex | Average* | deviation | subjects |
| G-1695A | G/G | F | 1.039 | 1.311 | 26 |
| | | M | 1.121 | 1.111 | 33 |
| | | Sum | 1.085 | 1.193 | 59 |
| | G/A | F | 0.724 | 1.066 | 29 |
| | | M | 0.732 | 0.895 | 41 |
| | | Sum | 0.729 | 0.962 | 70 |
| | A/A | F | 3.000 | 4.359 | 3 |
| | | M | 1.286 | 1.380 | 7 |
| | | Sum | 1.800 | 2.486 | 10 |
| IVS + C266G | C/C | F | 1.018 | 1.521 | 55 |
| | | M | 0.921 | 1.055 | 76 |
| | | Sum | 0.962 | 1.267 | 131 |
| | C/G | F | 0.333 | 0.577 | 3 |
| | | M | 1.250 | 0.957 | 4 |
| | | Sum | 0.857 | 0.900 | 7 |
| | G/G | M | 1.000 | — | 1 |
| | | Sum | 1.000 | — | 1 |
| TGA + C63T | C/C | F | 1.000 | 1.500 | 57 |
| | | M | 0.924 | 1.047 | 79 |
| | | Sum | 0.956 | 1.252 | 136 |
| | C/T | F | 0.000 | — | 1 |
| | | M | 1.500 | 0.707 | 2 |
| | | Sum | 1.000 | 1.000 | 3 |

| | | | | |
|---|---|---|---|---|
| Sum | F | 0.983 | 1.493 | 58 |
| | M | 0.938 | 1.041 | 81 |
| | Sum | 0.957 | 1.245 | 139 |

*Required number of analgesic doses during the 24 hour period following surgery.

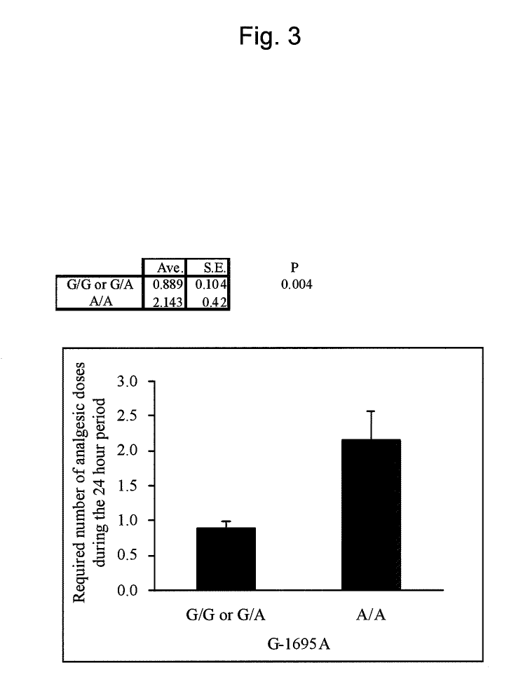

As shown in Table 6 below and FIG. 3, the patient group without the G allele of the G-1695A polymorphism required a significantly greater number of analgesic doses during the 24 hour period following surgery compared with the patient group with the G allele (P=0.004).

TABLE 6
Effect of POMC G-1695A polymorphism on the number of
analgesic doses during the 24 hour period following surgery
for patients receiving analgesics upon surgery (descriptive
statistics for each genotype and sex)

| POMC | | | | Number of |
|---|---|---|---|---|
| G-1695A | Sex | Average* | Standard deviation | test subjects |
| G/G or G/A | F | 0.872 | 1.187 | 55 |
| | M | 0.905 | 1.001 | 74 |
| | Sum | 0.892 | 1.084 | 129 |
| A/A | F | 3.000 | 4.359 | 3 |
| | M | 1.286 | 1.380 | 7 |
| | Sum | 1.800 | 2.486 | 10 |
| Sum | F | 0.983 | 1.493 | 58 |
| | M | 0.938 | 1.041 | 81 |
| | Sum | 0.957 | 1.245 | 139 |

*Required number of analgesic doses during the 24 hour period following surgery.

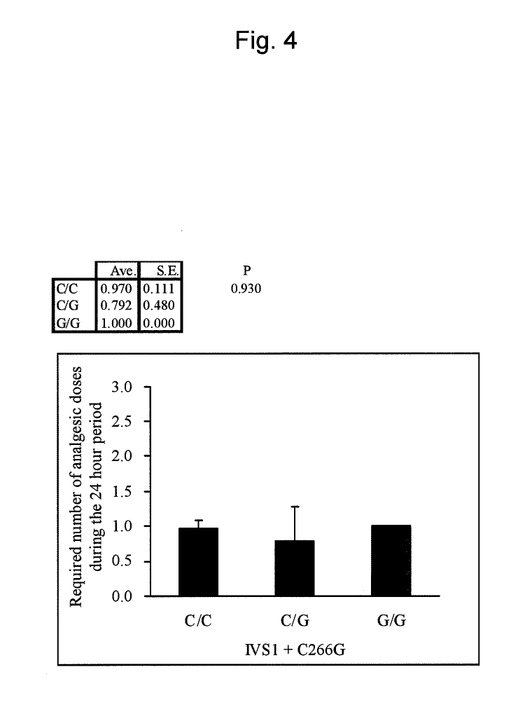

However, as shown in Table 5 above and FIG. 4, no significant difference was observed in the required number of analgesic doses during the 24 hour period following surgery among the patients with the C/C, C/G, and G/G genotypes of the IVS1+C266G polymorphism (P=0.930).

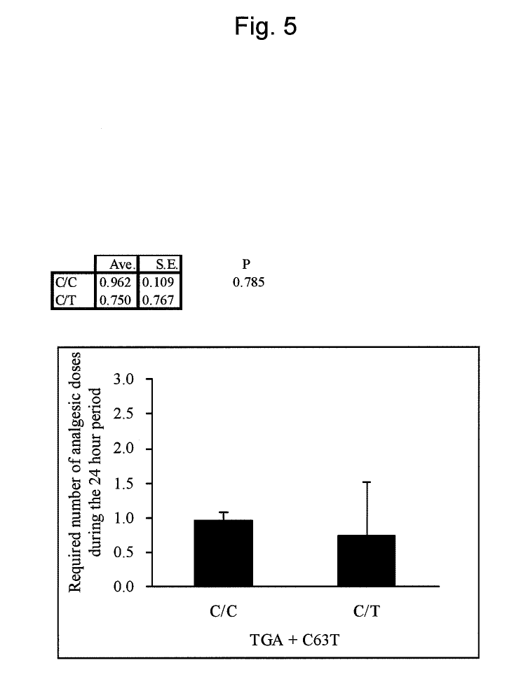

Additionally, as shown in Table 5 above and FIG. 5, no significant difference was observed in the required number of analgesic doses during the 24 hour period following surgery among patients with the C/C and C/T genotypes of the TGA+C63T polymorphism (P=0.785).

From these results, the number of analgesic doses required was proven to be readily predicted by determining the genotype of the POMC genetic polymorphism G-1695A.

### Example 2

### Association Between POMC Genetic Polymorphisms and Total Amount of Analgesics

Genomic DNA was extracted from blood or mucosa of the oral cavity of 139 patients who underwent surgery to determine the genotype of a genetic polymorphism (G-1695A) of the POMC gene by the PCR-RFLP method with the primer sets 1-3 shown in Table 3 of EXAMPLE 1 (SEQ ID NOS:7-12). Additionally, the primers shown in Table 4 of EXAMPLE 1 (SEQ ID NOS:13-16) were used to determine the genotypes of IVS1+C266G and TGA+C63T polymorphisms of the POMC gene by a sequencing procedure. Associations between the results of determining the genotypes of these genetic polymorphisms and the total amount of analgesics after surgery were analyzed.

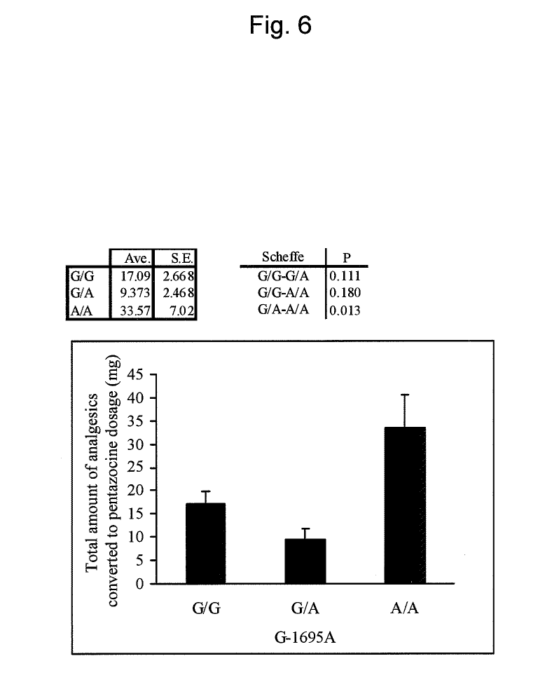

As a result, as shown in Table 7 below and FIG. 6, the patient group with the A/A genotype of the G-1695A polymorphism had a significantly greater total amount of analgesics converted to pentazocine-equivalent dosages compared with the patient group with G/A. (P=0.013). In particular, females with the A/A genotype tended to have a greater total amount of analgesics converted to pentazocine-equivalent dosages. In this regard, the phrase “total amount of analgesics converted to pentazocine-equivalent dosages” refers to a total amount of analgesics (milligrams) in which the amount of each of the administered analgesics is converted to an equivalent amount of pentazocine.

TABLE 7
Effects of POMC genetic polymorphisms on total amount of
analgesics converted to pentazocine-equivalent dosages for
patients receiving analgesics upon surgery
(descriptive statistics for each genotype and sex)

| | | | | | Number |
|---|---|---|---|---|---|
| POMC genetic | | | | Standard | of test |
| polymorphism | Genotype | Sex | Average* | deviation | subjects |
| G-1695A | G/G | F | 17.596 | 27.978 | 26 |
| | | M | 16.591 | 18.633 | 33 |
| | | Sum | 17.034 | 23.004 | 59 |
| | G/A | F | 9.052 | 13.942 | 29 |
| | | M | 9.695 | 13.546 | 41 |
| | | Sum | 9.429 | 13.615 | 70 |
| | A/A | F | 42.500 | 61.084 | 3 |
| | | M | 24.643 | 26.237 | 7 |
| | | Sum | 30.000 | 36.912 | 10 |
| IVS + C266G | C/C | F | 15.272 | 25.616 | 55 |
| | | M | 13.322 | 17.129 | 76 |
| | | Sum | 14.141 | 21.042 | 131 |
| | C/G | F | 2.500 | 4.330 | 3 |
| | | M | 22.500 | 25.981 | 4 |
| | | Sum | 13.929 | 21.402 | 7 |
| | G/G | M | 15.000 | — | 1 |
| | | Sum | 15.000 | — | 1 |
| TGA + C63T | C/C | F | 14.868 | 25.255 | 57 |
| | | M | 13.196 | 16.866 | 79 |
| | | Sum | 13.897 | 20.727 | 136 |
| | C/T | F | 0.000 | — | 1 |
| | | M | 37.500 | 31.820 | 2 |
| | | Sum | 25.000 | 31.225 | 3 |

| | | | | |
|---|---|---|---|---|
| Sum | F | 14.612 | 25.109 | 58 |
| | M | 13.796 | 17.447 | 81 |
| | Sum | 14.137 | 20.905 | 139 |

*Total amount of analgesics (milligrams) converted to pentazocine-equivalent dosages.

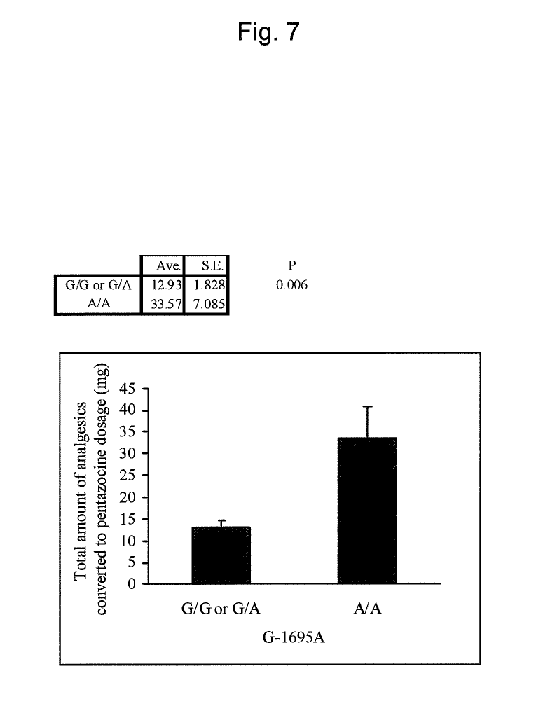

Moreover, as shown in Table 8 below and FIG. 7, the patient group without the G allele of the G-1695A polymorphism had a significantly greater total amount of analgesics converted to pentazocine-equivalent dosages compared with the patient group with the G allele (P=0.006).

TABLE 8
Effect of POMC G-1695A polymorphism on total amount of
analgesics converted to pentazocine-equivalent dosages for
patients receiving analgesics upon surgery
(descriptive statistics for each genotype and sex)

| POMC | | | | Number of |
|---|---|---|---|---|
| G-1695A | Sex | Average* | Standard deviation | test subjects |
| G/G or G/A | F | 13.091 | 21.948 | 55 |
| | M | 12.770 | 16.268 | 74 |
| | Sum | 12.907 | 18.820 | 129 |
| A/A | F | 42.500 | 61.084 | 3 |
| | M | 24.643 | 26.237 | 7 |
| | Sum | 30.000 | 36.912 | 10 |
| Sum | F | 14.612 | 25.109 | 58 |
| | M | 13.796 | 17.447 | 81 |
| | Sum | 14.137 | 20.905 | 139 |

*Total amount of analgesics (milligrams) converted to pentazocine-equivalent dosages.

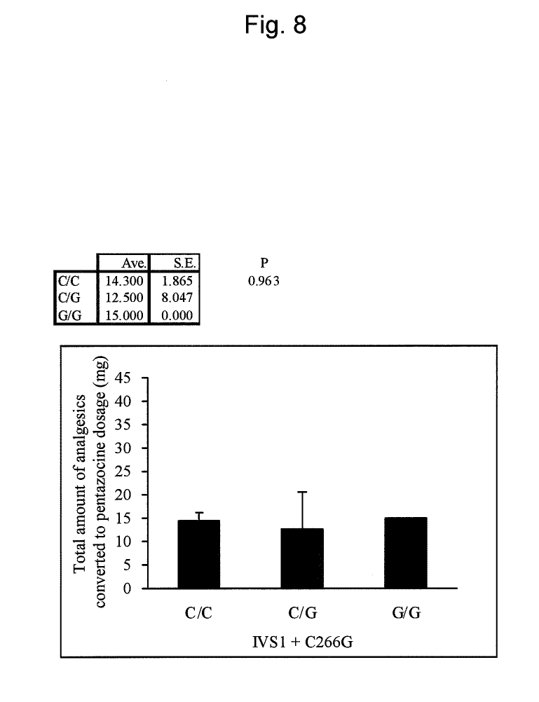

However, as shown in Table 7 above and FIG. 8, no significant difference was observed in the total amount of analgesics converted to pentazocine-equivalent dosages among the patients with the C/C, C/G, and G/G genotypes of the IVS1+C266G polymorphism (P=0.963).

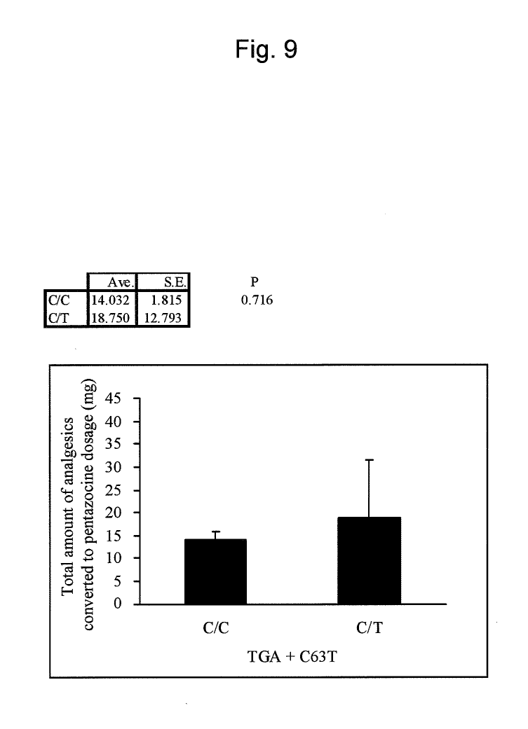

Additionally, as shown in Table 7 above and FIG. 9, no significant difference was observed in the total amount of analgesics converted to pentazocine-equivalent dosages among the patients with the C/C and C/T genotypes of the TGA+C63T polymorphism (P=0.716).

From these results, the total amount of analgesics was proven to be readily predicted by determining the genotype of the POMC genetic polymorphism G-1695A.

### Example 3

### Association Between POMC Genetic Polymorphism (G-1695A) and Alcohol Sensitivity

Genomic DNA was extracted from blood of 425 alcoholic patients and 339 healthy subjects to determine the genotype of a genetic polymorphism (G-1695A) of the POMC gene by the PCR-RFLP method with the primer sets 1-3 shown in Table 3 of EXAMPLE 1 (SEQ ID NOS:7-12).

As a result, as shown in Table 9 below, the frequency of each of the G/G, G/A and A/A genotypes tended to differ between the alcoholic patients and the healthy subjects (P=0.072). Furthermore, as shown in Table 9 below, the G/G and G/A genotype frequencies were found to be significantly high in the alcoholic patients (P=0.024).

TABLE 9
Comparison of genotype frequencies of POMC G-1695A polymorphism
between healthy subjects and alcoholic patients

| SNP name | POMC G-1695A | | | | |
|---|---|---|---|---|---|
| (sample | Genotype frequency (%) | | | | |
| number) | G/G | G/A | A/A | G/G or G/A | A/A |
| Healthy | 178 | 122 | 39 | 300 | 39 |
| subjects | (52.5%) | (36.0%) | (11.5%) | (88.5%) | (11.5%) |
| (339) | | | | | |
| Alcoholic | 229 | 167 | 29 | 396 | 29 |
| patients | (53.9%) | (39.3%) | (6.8%) | (93.2%) | (6.8%) |
| (425) | | | | | |
| | | | X² = 5.254 | | X² = 5.096 |
| | | | P = 0.0723 | | P = 0.0240 |

### Example 4

### Association Between the POMC Genetic Polymorphism (G-1695A) Classified by ALDH2 Genotype and Alcohol Sensitivity

Genomic DNA was extracted from blood of 425 alcoholic patients and 339 healthy subjects to determine the genotype of a genetic polymorphism (G-1695A) of the POMC gene by the PCR-RFLP method with the primer sets 1-3 shown in Table 3 of EXAMPLE 1 (SEQ ID NOS:7-12).

As a result, as shown in Table 10 below, a significant difference was observed in the genotype frequencies between the alcoholic patients and the healthy subjects with the 1*1 genotype of the ALDH2 genotype (P=0.026). Moreover, as shown in Table 10 below, the G/G and G/A genotype frequencies were found to be significantly high in the alcoholic patients (P=0.012).

TABLE 10
Comparison of genotype frequencies of POMC G-1695A
polymorphism between healthy subjects and alcoholic
patients classified by ALDH2 genotype

| SNP name | | | | | | |
|---|---|---|---|---|---|---|
| | ALDH2 | POMC G-1695A | | | | |
| | genotype | Genotype frequency (%) | | | | |
| (sample | (sample | | | | G/G | |
| number) | number) | G/G | G/A | A/A | or G/A | A/A |
| Healthy | 1*1 | 103 | 64 | 27 | 167 | 27 |
| subjects | (194) | (53.1%) | (33.0%) | (13.9%) | (86.1%) | (13.9%) |
| (339) | 1*2 | 68 | 50 | 9 | 118 | 9 |
| | (127) | (53.5%) | (39.4%) | (7.1%) | (92.9%) | (7.1%) |
| | 2*2 | 7 | 8 | 3 | 15 | 3 |
| | (18) | (38.9%) | (44.4%) | (16.7%) | (83.3%) | (16.7%) |
| Alcoholic | 1*1 | 201 | 151 | 28 | 352 | 28 |
| patients | (380) | (52.9%) | (39.7%) | (7.4%) | (92.6%) | (7.4%) |
| (425) | 1*2 | 28 | 16 | 1 | 44 | 1 |
| | (45) | (62.2%) | (35.6%) | (2.2%) | (97.8%) | (2.2%) |

### Example 5

### Association Between POMC Genetic Polymorphism (G-1695A) Classified by ADH2 Genotype and Alcohol Sensitivity

Genomic DNA was extracted from blood of 425 alcoholic patients and 339 healthy subjects to determine the genotype of a genetic polymorphism (G-1695A) of the POMC gene by the PCR-RFLP method with the primer sets 1-3 shown in Table 3 of EXAMPLE 1 (SEQ ID NOS:7-12).

As a result of comparisons between the alcoholic patients and the healthy subjects with the 1*2 genotype of ADH2 genotype, G/G and G/A genotype frequencies were found to be significantly high in the alcoholic patients (P=0.046) as shown in Table 11 below.

TABLE 11
Comparison of genotype frequencies of POMC G-1695A
polymorphism between healthy subjects and alcoholic patients
classified by ADH2 genotype

| SNP name | | | | | | |
|---|---|---|---|---|---|---|
| | ADH2 | POMC G-1695A | | | | |
| | genotype | Genotype frequency (%) | | | | |
| (sample | (sample | | | | G/G | |
| number) | number) | G/G | G/A | A/A | or G/A | A/A |
| Healthy | 1*1 | 13 | 7 | 1 | 20 | 1 |
| subjects | (21) | (61.9%) | (33.3%) | (4.8%) | (95.2%) | (4.8%) |
| (339) | 1*2 | 67 | 45 | 17 | 112 | 17 |
| | (129) | (51.9%) | (34.9%) | (13.2%) | (86.8%) | (13.2%) |
| | 2*2 | 98 | 70 | 21 | 168 | 21 |
| | (189) | (51.9%) | (37.0%) | (11.1%) | (88.9%) | (11.1%) |
| Alcoholic | 1*1 | 58 | 58 | 7 | 116 | 7 |
| patients | (123) | (47.2%) | (47.2%) | (5.7%) | (94.3%) | (5.7%) |
| (425) | 1*2 | 81 | 55 | 9 | 136 | 9 |
| | (145) | (55.9%) | (37.9%) | (6.2%) | (93.8%) | (6.2%) |
| | 2*2 | 90 | 54 | 13 | 144 | 13 |
| | (157) | (57.3%) | (34.4%) | (8.3%) | (91.7%) | (8.3%) |

### Example 6

### Association Between POMC Genetic Polymorphism (G-1695A) and Methamphetamine sensitivity

Genomic DNA was extracted from blood of 164 stimulant-type drug (methamphetamine)-dependent patients to determine the genotype of a genetic polymorphism (G-1695A) of the POMC gene by the PCR-RFLP method with the primer sets 1-3 shown in Table 3 of EXAMPLE 1 (SEQ ID NOS:7-12).

As a result, as shown in Table 12 below, when the patient group who underwent early withdrawal and experienced stimulant psychosis (less than 1 month) were compared with the patient group with prolonged stimulant psychosis (1 month or longer), G/G and G/A genotype frequencies were found to be significantly high in the patient group with prolonged delusion and hallucination (P=0.072).

TABLE 12
Comparison of genotypes of POMC G-1695A polymorphism in methamphetamine-
dependent patients classified according to duration of delusions and hallucinations

| SNP name | | | | | |
|---|---|---|---|---|---|
| Methamphetamine-dependent | POMC G-1695A | | | | |
| patients | Genotype frequency (%) | | | | |
| (sample number) | G/G | G/A | A/A | G/G or G/A | A/A |
| With early withdrawal | 53 | 38 | 8 | 91 | 8 |
| comprising delusions and | (53.5%) | (38.4%) | (8.1%) | (91.9%) | (8.1%) |
| hallucinations | | | | | |
| (99) | | | | | |
| With prolonged delusions and | 32 | 32 | 1 | 64 | 1 |
| hallucinations | (49.2%) | (49.2%) | (1.5%) | (98.5%) | (1.5%) |
| (65) | | | | | |
| | | | X² = 4.283 | | X² = 3.238 |
| | | | P = 0.1175 | | P = 0.0720 |

The results from Examples 3-6 above proved that the determination of the genotype of a POMC polymorphism, G-1695A, also allows easy prediction of drug sensitivity relative to vulnerability to drug addiction.

The present invention provides a genetic polymorphism in the 5′ flanking region of the POMC gene that allows evaluation of the difference in drug sensitivity among individuals and a method for evaluating drug sensitivity by using the genetic polymorphism. Use of this evaluation method allows one to easily determine an optimal dosage to be prescribed and an optimal schedule and the like associated with a narcotic analgesic, such as morphine, which is extremely beneficial for personalized pain therapy and treatment of drug dependence.

### SEQUENCE LISTING FREE TEXT

SEQ ID NO:7: Synthetic DNA

SEQ ID NO:8: Synthetic DNA

SEQ ID NO:9: Synthetic DNA

SEQ ID NO:10: Synthetic DNA

SEQ ID NO:11: Synthetic DNA

SEQ ID NO:12: Synthetic DNA

SEQ ID NO:13: Synthetic DNA

SEQ ID NO:14: Synthetic DNA

SEQ ID NO:15: Synthetic DNA

SEQ ID NO:16: Synthetic DNA

1. A method for evaluating drug sensitivity, comprising associating a genetic polymorphism in the 5′ flanking region of a POMC gene with an individual drug sensitivity.

2. A method according to claim 1, wherein the drug is an opioid receptor function-modifying agent.

3. A method according to claim 2, wherein the opioid receptor-modifying agent is at least one selected from the group consisting of methamphetamine, methylenedioxymethamphetamine, amphetamine, dextroamphetamine, dopamine, cocaine, heroin, morphine, hydromorphone, oxymorphone, DAMGO, codeine, dihydrocodeine, methadone, oxycodone, hydrocodone, fentanyl, carfentanil, tramadol, meperidine, dextropropoxyphene, dextromoramide, deltorphin, DPDPE, DADLS, DSTLE, DAMEA, U50488H, ketocyclazocine, enadoline, bremazocine, levorphanol, etorphine, dihydroetorphine, buprenorphine, nalorphine, nalbuphine, pentazocine, levallorphan, butorphanol, naltrexone, alvimopan, naltrindole, norbinaltorphimine (nor-BNI), Mr2266, naloxone, methanol, ethanol, propanol, butanol, pentanol, and diethyl ether.

4. A method according to claim 1, wherein the genetic polymorphism is at least one selected from the group consisting of a single nucleotide polymorphism, an insertion polymorphism, a deletion polymorphism, and a nucleotide repeat polymorphism.

5. A method according to claim 1, wherein the rs number of the genetic polymorphism is rs3754860.

6. A method for determining the type, the number of doses, and/or the dosage of a drug to be administered to an individual, comprising using the results from the evaluation by the method according to claim 1 as an index.

7. A method for predicting a side effect of a drug to be administered to an individual, comprising using the results from the evaluation by the method according to claim 1 as an index.

8. An oligonucleotide comprising a nucleotide sequence of at least 10 nucleotides comprising the 51st nucleotide of the nucleotide sequence represented by SEQ ID NO:1 or 2, or a complementary nucleotide sequence thereto.

9. An oligonucleotide according to claim 8, wherein the oligonucleotide spans a length of 10 to 45 nucleotides.

10. An oligonucleotide comprising a nucleotide sequence represented by SEQ ID NO:1 or 2, or a complementary nucleotide sequence thereto.

11. A microarray comprising the oligonucleotide according to claim 8 immobilized on a support.

12. A kit for evaluating drug sensitivity, comprising the oligonucleotide according to claim 8.

13. A kit for evaluating drug sensitivity, comprising the oligonucleotide according to claim 10.

14. A kit for evaluating drug sensitivity, comprising the microarray according to claim 11.

15. A microarray comprising the oligonucleotide according to claim 10 immobilized on a support.


SEQUENCE LISTING (16 sequences)

SEQ ID NO: 1 - DNA, 101 bp, Homo Sapiens
accaaaacccaaaattagctgggcatggtggcatgcatctgtggttccagrtactcaggaggctgaagcatgaggatcacttgagcccaggaggtggaggc

SEQ ID NO: 2 - DNA, 101 bp, Homo Sapiens
gcctccacctcctgggctcaagtgatcctcatgcttcagcctcctgagtayctggaaccacagatgcatgccaccatgcccagctaattttgggttttggt

SEQ ID NO: 3 - DNA, 101 bp, Homo Sapiens
ccgcgaagctgcaggcgctgtctccagggagccggcggcctcctctccccsaggggctcgcggcggtccggaggctccgagagcttgctaggaggtcttgg

SEQ ID NO: 4 - DNA, 101 bp, Homo Sapiens
ccaagacctcctagcaagctctcggagcctccggaccgccgcgagcccctsggggagaggaggccgccggctccctggagacagcgcctgcagcttcgcgg

SEQ ID NO: 5 - DNA, 101 bp, Homo Sapiens
gccccagggctaccctcccccaggaggtcgaccccaaagccccttgctctyccctgccctgctgccgcctcccagcctggggggtcgtggcagataatcag

SEQ ID NO: 6 - DNA, 101 bp, Homo Sapiens
ctgattatctgccacgaccccccaggctgggaggcggcagcagggcagggragagcaaggggctttggggtcgacctcctgggggagggtagccctggggc

SEQ ID NO: 7 - DNA, 21 bp, Artificial
  synthetic DNA
ccagtgattctaaatgcagtt

SEQ ID NO: 8 - DNA, 21 bp, Artificial
  synthetic DNA
gtaacttcaggaggctgctgg

SEQ ID NO: 9 - DNA, 21 bp, Artificial
  synthetic DNA
ccagtgattctaaatgcagtt

SEQ ID NO: 10 - DNA, 23 bp, Artificial
  synthetic DNA
gaattagggtctcactctgttgc

SEQ ID NO: 11 - DNA, 21 bp, Artificial
  synthetic DNA
ccagtgattctaaatgcagtt

SEQ ID NO: 12 - DNA, 24 bp, Artificial
  synthetic DNA
gcagatggtggttttgccttattt

SEQ ID NO: 13 - DNA, 20 bp, Artificial
  synthetic DNA
agcctcccgagacaggtaag

SEQ ID NO: 14 - DNA, 20 bp, Artificial
  synthetic DNA
cctgttgtcgggagtttgag

SEQ ID NO: 15 - DNA, 18 bp, Artificial
  synthetic DNA
cccaaggacaagcgctac

SEQ ID NO: 16 - DNA, 20 bp, Artificial
  synthetic DNA
ccatgctgctgttatttgac